(19) United States
(12) Patent Application Publication
(10) Pub. No.: US 2006/0276805 A1
(43) Pub. Date: Dec. 7, 2006

(54) Vessel recanalizer

(76) Inventor: Chun Ho Yu, Hong Kong (HK)

Correspondence Address: Raymond Y. Chan, #128, 108 N. Ynez Ave., Monterey Park, CA, 91754, US

(21) Appl. No.: 11/121,848

(22) Filed: May 3, 2005

(51) Int. Cl. A61B 17/26

(52) U.S. Cl. 606/114

(57) ABSTRACT

A vessel recanalizer, for removing a blood clog in a blood vessel, includes an elongated main wire, and a capturing pocket. When the main wire is in vicinity with the blood clog, the main wire is actuated to open the capturing pocket for capturing the blood clog therein. The capturing pocket is then closed to retain the blood clog within the capturing pocket. By pulling the main wire outwards, the blood clog is then removed from the blood vessel along with the capturing pocket.

### BACKGROUND OF THE PRESENT INVENTION

1. Field of Invention

The present invention relates to a vessel recanalizer, and more particularly to a vessel recanalizer which comprises a capturing pocket wire and an introducer catheter for removing a piece of blood clog within the blood vessel, so as to recanalize the blood vessel. Apart from blood clogs, the invention is also applicable for removal of dislodged cholesterol plaques within a blood vessel, and for removal of sludge or stone within a bile duct or urinary tract.

2. Description of Related Arts

Blood clogs in blood vessels are caused by the reduction in blood flow rate within a narrowed blood vessel or by migration of a blood clog which is formed within the heart.

It is well-known to the medical profession that blood clogs in blood vessels poses serious health problems, yet are very difficult to get rid of. Currently, blood clogs and blood vessels blockage conditions are generally cured by the procedures below.

The first procedure is that a catheter is guided into a blood vessel with a small balloon connected to the end of the catheter. The small balloon is then inflated after the end of the catheter has passed through the piece of blood clog. The wire is then retrieved with the inflated balloon pulling the piece of blood clog out of the blood vessel. The second is that a device is inserted into the blood vessel to suck the piece of blood clog out of the blood vessel. The third is that a device is inserted into the blood vessel to break the piece of blood clog into tiny pieces.

The first procedure has the problem of requiring surgery to cut open the vessel to insert the balloon and to take out the inflated balloon and to retrieve blood clog through the open wound.

The device used for sucking up blood clogs out of the blood vessel may or may not be able to remove blood clogs effectively. However, blood is usually sucked out along with the blood clogs. As a result, the patient may suffer a substantial amount of blood loss, which may be dangerous to the patient. Moreover, the suction tube is introduced directly into the blood vessel through a percutaneous puncture and therefore the size of the tube is very limited and usually significantly smaller than that of the blood clog, so much so that the blood clog could not be removed effectively.

The device for the third procedure, with which the piece of blood clog is broken into pieces, may often fail to break the blood clog, rendering the procedure ineffective. Even when the piece of blood clog is actually broken up into tiny pieces, they may still not be able to pass through the blood vessel and remain in the blood vessel. The procedure may even damage the blood vessel or may block off smaller vessels downstream.

There are also other methods for filtering out free floating blood clog particles during the procedure of widening narrowed vessels for preventing ischemia of target organs such as the heart and the brain, and preventing such free floating particles from flowing with the blood to target organs to cause ischemic complications, but these methods do not actively remove the blood clog at a specific location in the blood vessel.

As a result, in order to better help patients with the conditions of blood clogs and blood vessels blockage, so as to minimize their risks of suffering serious health conditions, a more effective and efficient device and method for actively removing blood clogs from major arteries leading to important organs, and also arteries and veins must be provided.

In cases of biliary sludge or biliary stone removal, the current methods are: (1) using a basket-like device; (2) using a biliary tract endoscope; and (3) open surgery. The basket device can be introduced into the biliary system percutaneously to trap the stone and take it out percutaneously, or be introduced from the gut through an endoscope to trap the stone and pull it down into the gut. For stone removal through a biliary tract endoscope, a percutaneous tract has to be prepared by serial dilatation over a few weeks, it is therefore a very time consuming procedure. For open surgery, the patient has to have the abdomen and the liver cut open to gain access to the bile duct.

### SUMMARY OF THE PRESENT INVENTION

A main object of the present invention is to provide a vessel recanalizer which comprises an introducer catheter, a main wire and a capturing pocket for removing a blocking object within a tubular structure of the human body, such as a piece of blood clog within the blood vessel, or a stone within the bile duct, so as to resume the patency of the tubular structure.

Another object of the present invention is to provide a vessel recanalizer that can capture the blocking object in the tubular structure when the opening of the capturing pocket has passed the blocking object, such that the blocking objects within the tubular structure are completely, effectively and actively captured.

Another object of the present invention is to provide a vessel recanalizer which has at least one restoring ring mounted at the opening of the capturing pocket, such that the capturing pocket is compressed within the lumen of the introducer catheter until the introducer catheter passes the blocking object, when the introducer catheter is slid backwards with the main wire held in position, such that the restoring ring erects and opens up the capturing pocket.

Another object of the present invention is to provide a vessel recanalizer which can remove blocking objects from and reopens the tubular structures without having to break the blocking objects into tiny pieces, eliminating the possibility of leaving behind tiny and non-captured pieces in the tubular structures.

Another object of the present invention is to provide a vessel recanalizer, wherein the capturing pocket is retracted after the blood clog is captured therein so as to prevent the blood clog from be escaping accidentally.

Another object of the present invention is to provide a vessel recanalizer, wherein the capturing pocket has a peripheral portion having an elongated segment with a tapering diameter, such that the piece of blocking object captured within the capturing pocket flows towards the peripheral portion of the capturing pocket while the main wire with the capturing pocket is backwardly slid against the introducer catheter.

Another object of the present invention is to provide a vessel recanalizer, wherein the capturing pocket is made of porous material for allowing the blood or other body fluid to flow through the capturing pocket, and for ensuring that the blocking object remains in the capturing pocket.

Another object of the present invention is to provide a vessel recanalizer which effectively locates and removes the piece of blocking object from the tubular structure inside the body, such that the piece of blocking object is successfully removed from the tubular structure without unnecessarily removing excess blood or body fluid from the tubular structure, thereby successfully reopens the tubular structure for normal flow of blood or any other body fluid.

Another object of the present invention is to provide a vessel recanalizer which effectively, efficiently and actively removes blood clogs from major arteries leading to important organs, and also arteries and veins.

Another object of the present invention is to provide a vessel recanalizer, further comprises an introducer catheter provided for slidably receiving the main wire therein, so as to minimize the profile of the capturing pocket before it opens, for gaining access to the blocking object, and to accurately locate, as well as to guide the capturing pocket to a close vicinity of the blocking object in the tubular structure.

Another object of the present invention is to provide a vessel recanalizer, wherein the guiding end of the main wire has a guiding curve, so as to better guide the main wire and the introducer catheter through curves of the tubular structure, and to get through the blocking object.

Another object of the present invention is to provide a vessel recanalizer, wherein the restoring ring is made by a thermo sensitive material, such that the stiffness of the restoring ring increases when it is inserted into the body.

Another object of the present invention is to provide a method for removing blocking object in blood vessel by a vessel recanalizer, wherein the method accurately locates and removes the blocking object from the blood vessel, such that no excess blood or body fluid will be removed from the blood vessel, or leaving portions of the piece of blood clog in the blood vessel.

Accordingly, in order to accomplish the above objects, the present invention provides a vessel recanalizer for removing a piece of blocking object from a tubular structure, comprising:

an elongated main wire, which is sized and shaped for slidably extending along the blood vessel, having a control end and a guiding end adapted for reaching the blood clog in the blood vessel; and

a capturing pocket, which is provided at the main wire between the control end and the guiding end, located near the guiding end, having an opening facing against the control end and a receiving cavity having a size adapted for receiving the blood clog therein, wherein the capturing pocket is adapted to fold between an opened position that the opening of the capturing pocket is opened to communicate the receiving cavity with an exterior of the capturing pocket and a closed position that the opening of the capturing pocket is closed to enclose the receiving cavity, such that when the guiding end of the main wire is slid forwardly in vicinity of the blood clog, the capturing pocket opens to the opened position for aligning the opening with the piece of blood clog, such that the main wire is pulled backwardly for collecting the piece of blood clog in the receiving cavity so as to remove the blood clog in the blood vessel.

In an alternative embodiment, the present invention provides a method for removing a blocking object in blood vessel by a vessel recanalizer which comprises a main wire and a capturing pocket provided thereon, comprising the steps of:

(a) slidably inserting the vessel recanalizer along the blood vessel until the capturing pocket is positioned in vicinity of and has passed beyond the piece of blocking object;

(b) opening the capturing pocket and aligning an opening thereof towards the piece of blocking object;

(c) capturing the piece of blocking object in the capturing pocket;

(d) closing the opening of the capturing pocket to trap the piece of blocking object therein; and

(e) slidably ejecting the main wire from the blood vessel to remove the piece of blocking object therein.

These and other objectives, features, and advantages of the present invention will become apparent from the following detailed description, the accompanying drawings, and the appended claims.

### BRIEF DESCRIPTION OF THE DRAWINGS

FIG. 1 is a schematic perspective view of the vessel recanalizer according to the preferred embodiment of the present invention.

FIG. 2 is a schematic perspective view of the capturing pocket of the vessel recanalizer according to the above preferred embodiment of the present invention.

FIG. 3 is a schematic side view of the vessel recanalizer according to the above preferred embodiment of the present invention.

FIG. 4A to 4F illustrates schematic side views of the vessel recanalizer at different stages while it is used for removing a piece of blood clog in a blood vessel according to the above preferred embodiment of the present invention.

FIG. 5 is a flow chart illustrating the method of removing the piece of blood clog in the blood vessel according to the above preferred embodiment of the present invention.

### DETAILED DESCRIPTION OF THE PREFERRED EMBODIMENT

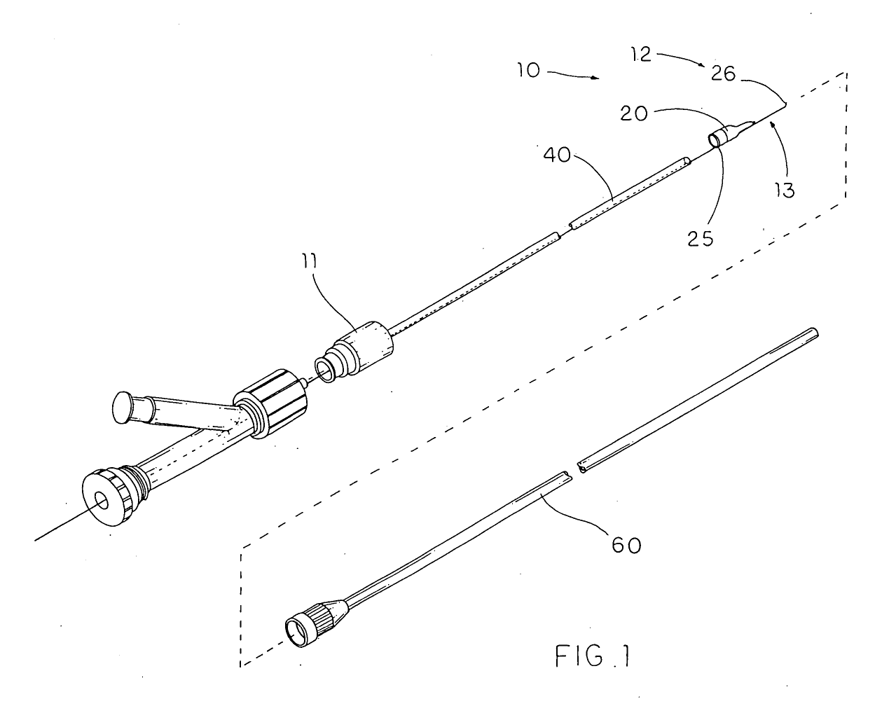

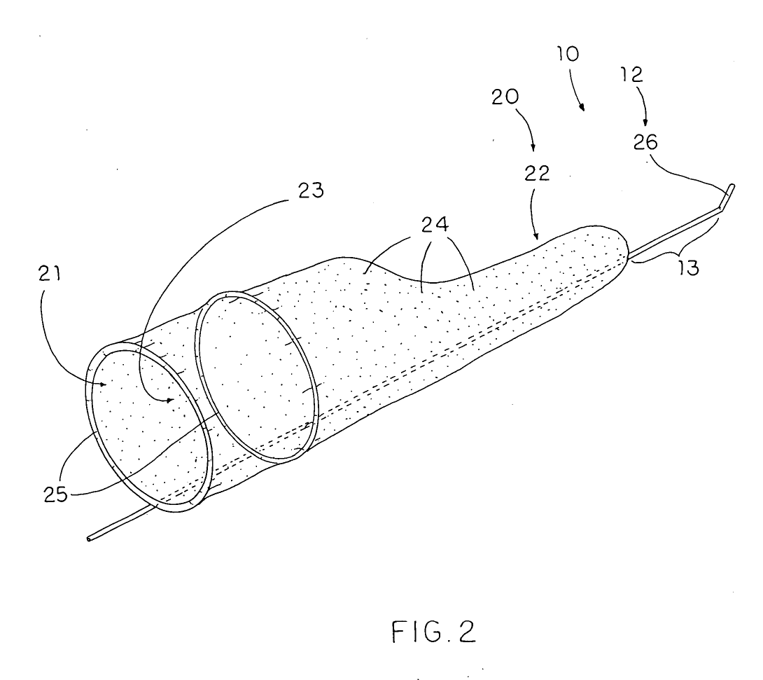

Referring to FIGS. 1 and 2 of the drawings, a vessel recanalizer for removing a piece of blocking object 2 in tubular structure 1 according to the present invention is illustrated, wherein the vessel recanalizer comprises an elongated main wire 10, a capturing pocket 20.

According to the preferred embodiment of the present invention, the vessel recanalizer is used for removing a piece of blood clog from a blood vessel. However, the vessel recanalizer can also be used for removing other blocking objects in other tubular structures of a body, such as stones in the biliary system or the urinary system, since the removal of such debris from such tubular structures are essentially the same as the removal of a piece of blood clog from a blood vessel.

The elongated main wire 10 is a flexible wire, provided for extending within the blood vessel 1 of a body, and more specifically, a human body. It must be made of a material such that slidable extension within the blood vessel 1 is possible, allowing an easy extension within the blood vessel 1 and easy manipulation of the main wire 10 while within the blood vessel 1.

In order to facilitate a successful extension into the blood vessel 1, the main wire 10 has a control end 11 provided for controlling the main wire 10 during its extension within the blood vessel 1 and while it is inside the blood vessel 1, and a guiding end 12 provided for guiding the main wire 10 through the blood vessel 1 to a desired location within the blood vessel 1.

The main wire 10 must have a substantial length so as to reach the piece of blood clog 2 successfully. It must also be small enough in width to be able to be fitted into the blood vessel 1, as well as the introducer catheter 40. If the width of the main wire 10 is too large, it may post a risk of bursting the blood vessel 1.

According to the preferred embodiment of the present invention, the main wire 10 has a width ranging from 0.01 inch to 0.018 inch, and a length of approximately 200 cm. However, the length and width may vary according to the blood vessel 1 size of the part of the body to which the vessel recanalizer is applied.

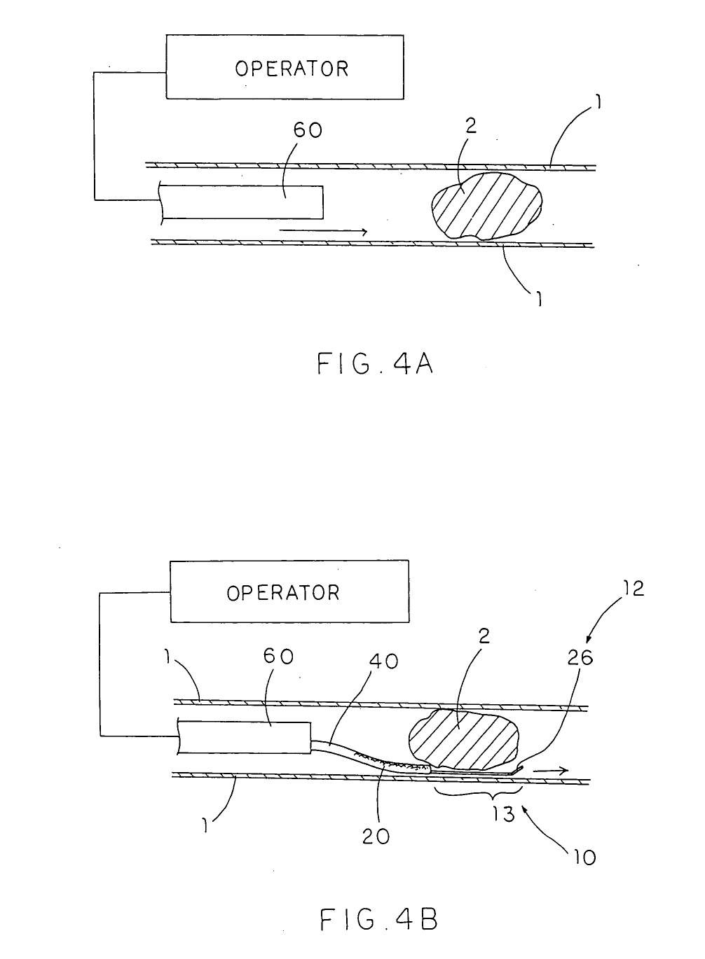

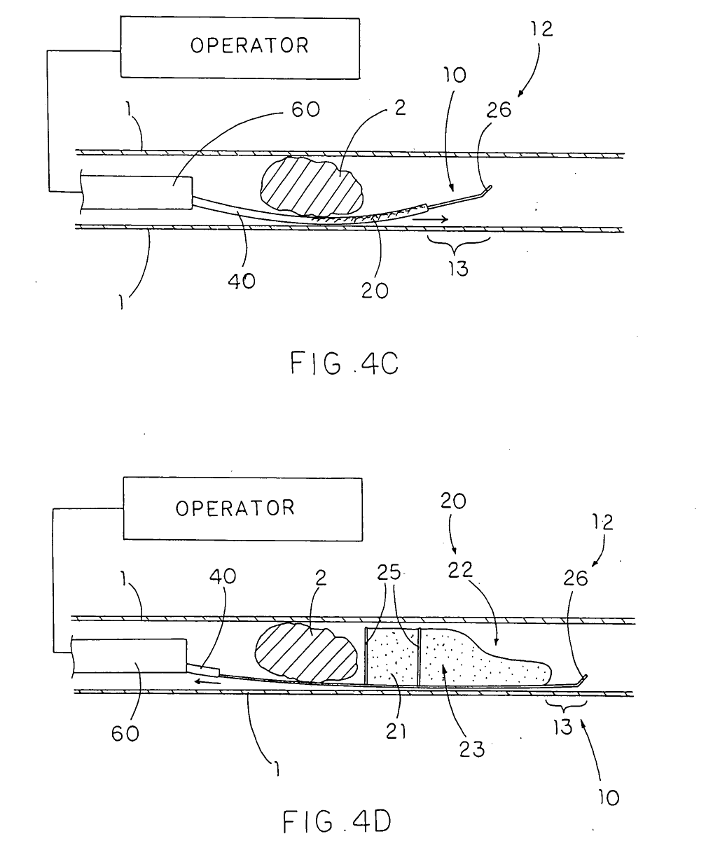

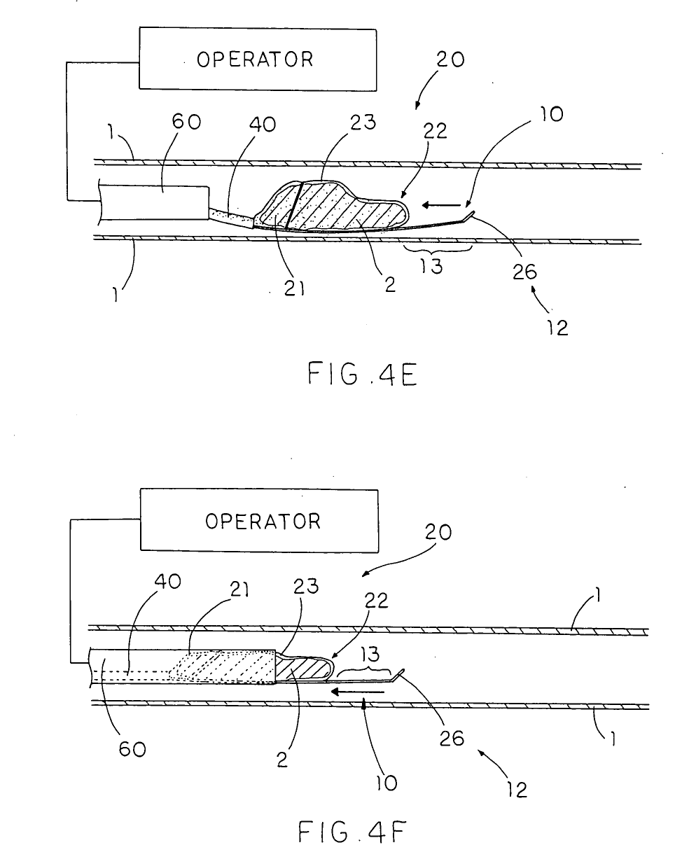

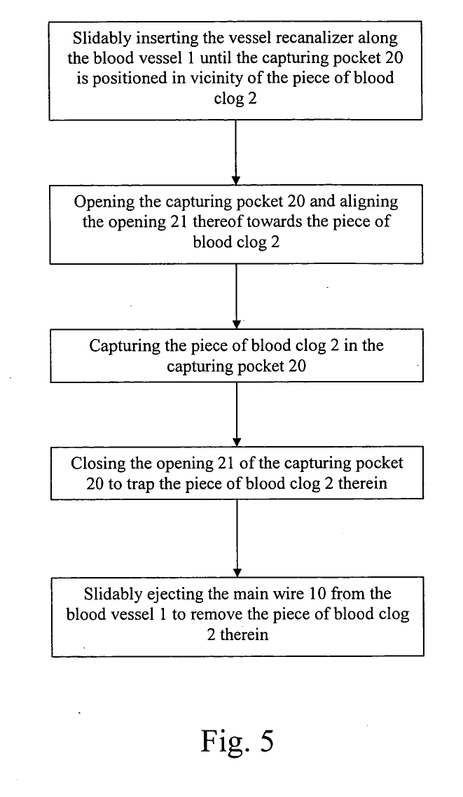

Hence, referring to FIG. 5 and FIG. 4A to 4C of the drawings, a first step of utilizing the vessel recanalizer to remove a piece of blood clog 2 from a blood vessel 1 is to slidably insert the vessel recanalizer along the blood vessel 1 until the capturing pocket 20 is positioned in vicinity of the piece of blood clog 2, wherein the main wire 10 and introducer catheter 40 together are slid along the blood vessel 1 as a single unit until the opening 21 of the capturing pocket 20 passes the piece of blood clog 2.

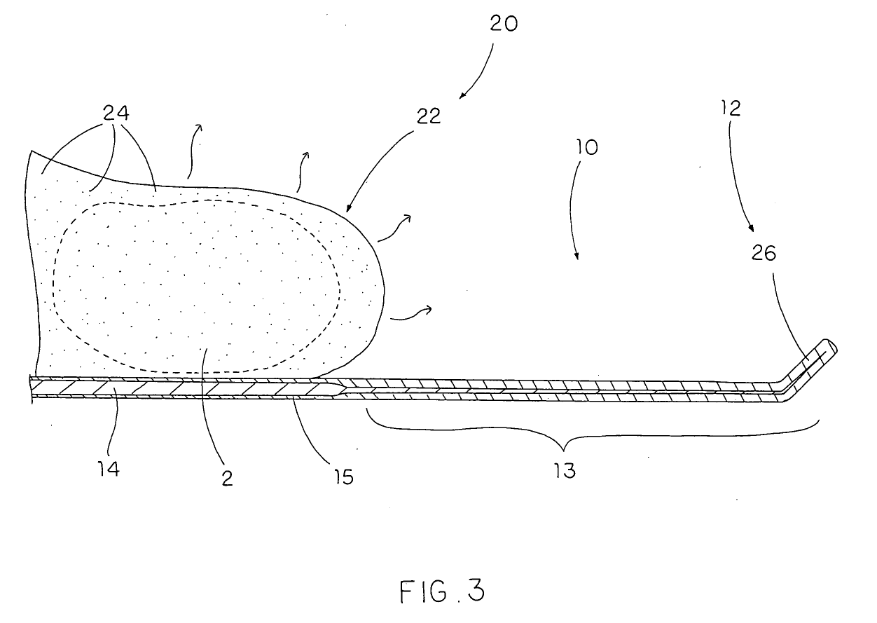

As shown in FIGS. 3 and 4, the capturing pocket 20 is provided on said main wire 10 between the control end 11 and the guiding end 12 of the main wire 10, and located near the guiding end 12 of the main wire 10, wherein the capturing pocket 20 has an opening 21, a peripheral portion 22, and a receiving cavity 23 of a substantial size for capturing and retaining the piece of blood clog 2 therein. When the main wire 10 is slidably extended within the blood vessel 1, the capturing pocket 20 moves along with the main wire 10 wherein the opening 21 of the capturing pocket 20 faces the control end 11 of the main wire 10.

For better guiding of the capturing pocket 20 to a close vicinity of the piece blood clog 2, it is provided close to the guiding end 12 of the main wire 10. It also prevents the capturing pocket 20 from detaching while the main wire 10 is retrieved.

For convenient extension of the main wire 10 into the blood vessel 1, the capturing pocket 20 is adapted to fold between the closed position and the opened position, wherein at the closed position, the opening 21 of the pocket is folded up in such a manner that the receiving cavity 23 is enclosed, and at the opened position, the opening 21 is unfolded in such a manner that the opening 21 is fully opened in such a manner that the receiving cavity 23 of the capturing pocket 20 communicates with an exterior of the capturing pocket 20.

When the vessel recanalizer is slidably extended into the blood vessel 1, the capturing pocket 20 is in a closed position, sitting within the lumen of the introducer catheter 40, such that the capturing pocket 20 will not be in an opened position creating turbulence or blockage in the blood flow. When the capturing pocket 20 is in the desired location, the opening 21 is opened, to open up the capturing pocket 20 in an opened position, allowing the receiving cavity 23 to receive blood or blood clog 2.

In order to better guide the capturing pocket 20 to a close vicinity of the piece of blood clog 2 in the blood vessel 1, the capturing pocket 20 is provided close to the guiding end of the main wire 10. Also, the capturing pocket 20 is securely attached to the main wire 10, so as to prevent the capturing pocket 20 from detaching while the main wire 10 is extended into the blood vessel 1, which may post danger to the patient.

Hence, referring to FIG. 5 and FIG. 4D of the drawings, a second step of utilizing the vessel recanalizer to remove a piece of blood clog 2 from a blood vessel 1 is to open the capturing pocket 20 and aligning the opening 21 of the capturing pocket 20 towards the piece of blood clog 2.

The folding of the capturing pocket 20 between the opened position and the closed position is controlled by an operator at the control end 11 of the main wire 10. When the introducer catheter 40 is slidably extended towards the guiding end 12 of the main wire 10, the capturing pocket 20 will be folded up in a closed position. When the introducer catheter 40 is slidably withdrawn towards the control end 11 of the main wire 10, the capturing pocket 20 will be opened up in an opened position.

When the guiding end 12 of the main wire 10 is in close vicinity of the piece of blood clog 2, the operator actuates the capturing pocket 20 to the opened position, such that the opening 21 is aligned with the piece of blood clog 2. The main wire is then pulled backwards to collect the piece of blood clog 2 in the receiving cavity 23 of the capturing pocket 20.

After the collection of the blood clog 2 in the capturing pocket 20 through the receiving cavity 23, the capturing pocket 20 is closed by slidably pulling the main wire 10 backwards against the introducer catheter 40 which is kept still. After the capturing pocket is closed, the main wire 10 and the introducer catheter 40 are removed from the blood vessel 1 together as a single unit, along with the capturing pocket 20 containing the piece of blood clog 2.

In order to render the capturing of the piece of blood clog 2 by the capturing pocket 20 successful, the opening 21 of the capturing pocket 20 must have a substantial opening width and a substantial pocket depth, allowing a piece of blood clog 2 of a general size to pass through the opening 21 and fit within the receiving cavity 23 of the capturing pocket 20.

According to the preferred embodiment of the present invention, the opening width of the opening 21 varies from 2 mm to 10 mm, and the depth of the capturing pocket 20 varies from 2 cm to 10 cm, depending on the location upon which the vessel recanalizer is applied. Obviously, if the vessel recanalizer is applied to a major artery, the opening width of the opening 21 and the depth of the capturing pocket 20 will be greater than that applied to a minor blood vessel.

Furthermore, the capturing pocket 20 is made of a porous material, wherein a plurality of pores 24 are provided on the capturing pocket 20, wherein the pores 24 are large enough for blood cells to pass through, such that blood entering the receiving cavity 23 of the capturing pocket 20 will exit the receiving cavity 23 of the capturing pocket 20 through the pores 24, without occupying the receiving cavity 23 which would hinder the capturing of the piece of blood clog 2.

However, the pores 24 are not large enough for the piece of blood clog 2 to pass though, such that while the blood cells exit the capturing pocket 20 through the pores 24, the piece of blood clog 2 would be trapped within the receiving cavity 23 of the pocket. In other words, the pores 24 allows the blood passing therethrough to ensure the blood clog 2 to be captured in the capturing pocket 20 when the capturing pocket 20 is slidably pulled backward.

Hence, referring to FIG. 5 of the drawings, a third step of utilizing the vessel recanalizer to remove a piece of blood clog 2 from a blood vessel 1 is to capture the piece of blood clog 2 in the capturing pocket 20, wherein the blood is guided to flow out of the capturing pocket through the pores 24 provided on the capturing pocket 20 when the piece of blood clog 2 is being captured in the capturing pocket 20.

And, referring to FIG. 4E of the drawings the forth step of utilizing the vessel recanalizer to remove a piece of blood clog 2 from a blood vessel 1 is to close the opening 21 of the capturing pocket 20 to trap the piece of blood clog 2 in it.

In addition, in order to render the piece of blood clog 2 successfully retained within the receiving cavity 23 of the capturing pocket 20, the capturing pocket 20 is constructed to have a tapered shape. The peripheral portion 22 of the capturing pocket 20 has an elongated segment with a tapering diameter in such a manner that the opening width of the opening 21 is larger than a bottom width of the peripheral portion 22, such that when the main wire 10 is removed from the blood vessel 1 by being slidably pulled backwards, the piece of blood clog 2 will be pushed towards and distributed into the peripheral portion 22 of the capturing pocket 20, such that the piece of blood clog 2 will be reinforced and trapped within the receiving cavity of the capturing pocket 20.

Hence, in order to better capture the piece of blood clog 2 in the capturing pocket, the piece of blood clog 2 is to be trapped at the peripheral portion 22 of the capturing pocket.

Hence, referring to FIG. 5 and FIG. 4F of the drawings, a fifth step of utilizing the vessel recanalizer to remove a piece of blood clog 2 from a blood vessel 1 is to slidably eject the main wire 10 from the blood vessel 1 to remove the piece of blood clog 2 from the blood vessel 1.

The vessel recanalizer according to the preferred embodiment of the present invention further comprises a introducer catheter 40, which slidably receives the main wire 10 and the capturing pocket 20 within it, in such a manner that when the capturing pocket 20 reaches the vicinity of the piece of blood clog 2 and passes beyond it, the introducer catheter 40 is being slid backwards, exposing the capturing pocket 20.

The introducer catheter 40 is provided to guide the main wire 10 and the capturing pocket 20 into the blood vessel 1, until the main wire 10 and the capturing pocket 20 are in close vicinity of the piece of blood clog 2, such that the capturing pocket 20 is protected within the introducer catheter 40 until it is required.

Also, by not exposing the main wire 10 and the capturing pocket 20 to the blood vessel 1 before the vessel recanalizer reaches to the vicinity of the piece of blood clog 2, the thin wire like main wire 10 is prevented from damaging the thin walls of the blood vessel 1, and also the capturing pocket 20 is prevented from accidentally opening up before reaching the piece of blood clog 2, in which case the opened capturing pocket 20 would be unable to pass through the blood clog 2 and would push the blood clog to a further downstream location.

The introducer catheter 40 of the vessel recanalizer must have a substantial length so as to reach the piece of blood clog 2 successfully. The width of the introducer catheter 40 must be small enough so as to avoid posting the risk of bursting the blood vessel 1.

According to the preferred embodiment of the present invention, the introducer catheter 40, having a length of approximately 150 cm and a diameter of 1 mm to 1.5 mm, is made of a flexible material, such that the vessel recanalizer can be directed towards the blood clog 2 in a much easier and safer manner, so as to prevent the vessel recanalizer from damaging the blood vessel 1.

It is worth mentioning that the length and width of the introducer catheter 40 may vary according to the size of the blood vessel, as well as other tubular structure of the part of the body to which the vessel recanalizer is applied.

In order to ensure that the opening 21 of the capturing pocket 20 is fully opened to reveal the receiving cavity 23 of the capturing pocket 20, the capturing pocket 20 further has at least one restoring ring 25 mounted at the opening 21 of the capturing pocket 20, wherein the restoring ring 25 is flexible, such that the restoring ring 25 expands to open the opening 21 of the capturing pocket 20 when the capturing pocket 20 is slid out of the introducer catheter 40, and contracts to close the opening 21 of the capturing pocket 20 when the capturing pocket 20 is slidably pulled back into the introducer catheter 40.

The restoring ring 25 is made of a thermo-sensitive material such that the restoring ring 25 remains soft below body temperature, and hardens up when the restoring ring 25 is inserted into the body along with the main wire 10.

According to the preferred embodiment of the present invention, the material making the restoring ring 25 is made a memory material, such as Nitinol, which remains soft at zero degrees Celsius and has metallic memory. The main wire 10 is cooled to 0° C. so that the restoring ring 25 softens, such that the capturing pocket 20 would collapse and allowing the capturing pocket 20 to fit within the introducer catheter 40 before being inserted into the blood vessel 1.

For convenient extension of the vessel recanalizer into the blood vessel 1, the capturing pocket 20 is first positioned inside the introducer catheter 40 in a closed position. When the vessel recanalizer is slidably extended into the blood vessel 1, the capturing pocket 20 remains in the closed position inside the introducer catheter 40, such that the capturing pocket 20 will not be in an open position. When the capturing pocket 20 is at the desired location, the main wire 10 is kept still to maintain the position of the capturing pocket 20, while the introducer catheter 40 is slid backwards and opens up the capturing pocket 20 into the opened position, allowing the capturing pocket 20 to receive blood or blood clog 2.

And, in order to open the capturing pocket 20, the introducer catheter 40 is slid backwards with the main wire 10 held in position wherein the capturing pocket 20 is slid out of the introducer catheter 40. Then, the restoring ring 25 erects at the opening of the capturing pocket 20 to fully unfold and open the capturing pocket 20 in such a manner that the capturing pocket 20 is ready for capturing.

Also, in order to close the opening of the capturing pocket 20 to trap the piece of blood clog 2, the main wire 10 is slidably pulled along the introducer catheter 40 in a backward direction. The restoring ring 25 then contracts to enclose the blood clog 2 in the capturing pocket 25.

When the capturing pocket 20 is slid out of the introducer catheter 40 along with the main wire 10, and reaches the body temperature inside the blood vessel 1, the restoring ring 25 would harden. And since Nitinol has metallic memory, when the capturing pocket 20 is extended into the blood vessel 1, the restoring ring 25 would return to its circular shape and open the opening 21 of the capturing pocket 20, exposing the receiving cavity 23 of the capturing pocket 20 and ensuring the blood clog 2 capturing ability of the capturing pocket 20.

Furthermore, according to the preferred embodiment of the present invention, the capturing pocket 20 has two restoring rings 25, where the distance between the two restoring rings 25 is approximately equal to the diameter of the restoring rings 25.

It is worth mentioning that in order to better guide the main wire 10 through a bend in the blood vessel 1, the guiding end 12 of the main wire 10 has a guiding curve 26 forms thereon. The guiding curve 26 is at a 45° angle to the main wire 10. The guiding curve 26 and a short length of the main wire 10 is exposed out of the introducer catheter 40 to provide better guiding of the main wire 10 and the introducer catheter 40 through curves or bends of the blood vessel 1.

When the main wire 10 reaches a bend of the blood vessel 1, the angle between the bending of the blood vessel 1 and the guiding curve 26 will be smaller than that between the bending of the blood vessel 1 and the main wire 10. As a result, the chance of damaging a blood vessel 1 by the main wire 10 is minimized. And the passage of the main wire 10 through the bend in the blood vessel 1 will be smoother.

Furthermore, the main wire 10 further has a floppy end portion 13 formed at the guiding end 12 towards the capturing pocket 20 for allowing the main wire 10 to extend along the blood vessel 1 until the capturing pocket 20 reaches the blood clog 2. In other words, the floppy end portion 13 of the main wire 10 is formed between the guiding end 12 of the main wire 10 and the position on the main wire 10 at the peripheral portion 22 of the capturing pocket 20, wherein the floppy end portion 13 is flexible, enhancing the curve passage ability of the main wire 10, increasing the usability and minimizing the risks of the vessel recanalizer.

According to the preferred embodiment of the present invention, the main wire 10 has an inner core 14 and an outer cover 15. The inner core 14 from the control end 11 to the capturing pocket 20 is made of either stainless steel or nitinol and the outer cover 15 is made of stainless steel. From the capturing pocket 20 to the guiding end 12, the inner core 14 is also made of stainless steel or nitinol but the outer cover 15 is either platinum or gold.

In order for the guiding end 12 of the main wire 10 to be flexible, the diameter of inner core 14 gradually decreases from the capturing pocket 20 to the guiding end 12. The reason of using platinum or gold is that platinum shows up under X-ray, allowing an operator of the control end 11 of the main wire 10 to know where the guiding end 12 is located.

It is also worth mentioning that before inserting the introducer catheter 40 and the main wire 10 into the blood vessel 1, the piece of blood clog 2 in the blood vessel 1 must be located, such that the vessel recanalizer can be accurately applied to the patient, so as to minimize risks posted on the patient.

Before the introducer catheter 40 is inserted into the blood vessel 1, the piece of blood clog 2 may be located externally by scanning the body. And, when the introducer catheter 40 is inserted into the blood vessel 1, but before the extension of the main wire 10 into the blood vessel 1, a monitoring device having a lens head extended along the blood vessel 1 to monitor the location of the introducer catheter 40 therein to further accurately determine the location of the piece of blood clog 2. In other words, the lens head, such as a camera or lens, can view the guiding end 12 of the main wire 10 in the blood vessel 1, such that the operator of the control end 11 of the main wire 10 can monitor the condition of the blood vessel 1 while the main wire 10 is slid in the blood vessel 1. The location of the blood clog 2 and the guiding end 12 can also be located and monitored by an electronic navigation system or by injection of dye under x-ray fluoroscopic control.

Referring to FIG. 1 and FIG. 3 of the drawings, according to the preferred embodiment, a retriever sheath 60 is used to slidably receive the introducer catheter 40 and the monitoring device therein such that the retriever sheath 60 can be slidably inserted into the blood vessel 1 until the retriever sheath 60 is located near the blood clog 2. The introducer catheter 40 and the monitoring device are then introduced through the retriever sheath 60 to work on the blood clog 2. As a result, the introducer catheter 40 must be small enough in width to be able to be fitted into the retriever sheath 60 and the blood vessel. The retriever sheath 60 can be used to deliver dye for localization of the blood clog 2 under x-ray fluoroscopy.

The operator, or most likely the surgeon, can control the sliding extension within the blood vessel 1 of the retriever sheath 60, the introducer catheter 40 and the main wire 10 with the capturing pocket 20 independently to a desired location within the blood vessel 1 through the manipulation at the end of the apparatus outside the patient's body. The monitoring of the location of blood clog 2, the vessel recanalizer and the entire surgery can be achieved through X-ray apparatus, electronic navigation system, or the camera attached to the introducer catheter 40.

The retriever sheath 60 is made of a flexible material with a diameter larger than the introducer catheter 40, wherein the retriever sheath 60 functions as a guiding conduit for easier insertion of the vessel recanalizer to be positioned near the piece of blood clog 2, as well as for receiving the capturing pocket 20 together with the retrieved blood clog before the blood clog is removed from the patent's body.

For a convenient extension of the vessel recanalizer into the blood vessel 1, it is first inserted through the retriever sheath 60 to reach a position close to the piece of blood clog 2.

According to the preferred embodiment of the present invention, the retriever sheath 60, having a length of approximately 60 cm to 100 cm and a diameter of 2 mm to 3 mm, is made of a flexible material, such that the vessel recanalizer can be inserted therein and reach the blood clog 2 in an easy and safe manner without damaging the blood vessel. However, the size of the retriever sheath 60 and the introducer sheath 40 may vary to suit the blocking object to be retrieved and the size of the tubular structure, for example, a larger vessel recanalizer is usually required for the removal of stones in uniary or biliary system.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A vessel recanalizer for removing a piece of blood clog in blood vessel, comprising:
an elongated main wire, which is sized and shaped for slidably extending along said blood vessel, having a control end and a guiding end adapted for reaching said blood clog in said blood vessel; and
a capturing pocket, which is provided at said main wire between said control end and said guiding end, and located near to the said guiding end, having an opening facing against said control end and a receiving cavity having a size adapted for receiving said blood clog therein, wherein said capturing pocket is adapted to fold between an opened position that said opening of said capturing pocket is opened to communicate said receiving cavity with an exterior of said capturing pocket and a closed position that said opening of said capturing pocket is closed to enclose said receiving cavity, such that when said guiding end of said main wire is slid forwardly in vicinity of said blood clog, said capturing pocket opens to said opened position for aligning said opening with said piece of blood clog, such that when said main wire is pulled backwardly, said receiving cavity collects said piece of blood clog in so as to remove said blood clog in said blood vessel.

2. The vessel recanalizer, as recited in claim 1, further comprising a introducer catheter slidably receiving said main wire therein, wherein said introducer catheter is slid backward against said main wire to open said capturing pocket when said capturing pocket is slid out of said introducer catheter.

3. The vessel recanalizer, as recited in claim 1, wherein said capturing pocket further comprises a restoring ring mounted at said opening, wherein said restoring ring having a flexibility expands a size thereof to open said opening of said capturing pocket when said capturing pocket is slid out of said introducer catheter and contracts said size to close said capturing pocket after said capturing pocket is slidably pulled back into said introducer catheter.

4. The vessel recanalizer, as recited in claim 2, wherein said capturing pocket further comprises a restoring ring mounted at said opening, wherein said restoring ring having a flexibility expands a size thereof to open said opening of said capturing pocket when said capturing pocket is slid out of said introducer catheter and contracts said size to close said capturing pocket after said capturing pocket is slidably pulled back into said introducer catheter.

5. The vessel recanalizer, as recited in claim 4, further comprises a retriever sheath slidably receiving said introducer catheter in such a manner that said retriever sheath is capable of slidably inserted into said blood vessel, and said introducer catheter is guided into said blood vessel therethrough.

6. The vessel recanalizer, as recited in claim 3, wherein said restoring ring is made of thermo sensitive material that said restoring ring increases in stiffness thereof at a body temperature.

7. The vessel recanalizer, as recited in claim 4, wherein said restoring ring is made of thermo sensitive material that said restoring ring increases in stiffness thereof at a body temperature.

8. The vessel recanalizer, as recited in claim 4, wherein said restoring ring has a metallic memory that said restoring ring returns to an original shape to open up said opening of said capturing pocket.

9. The vessel recanalizer, as recited in claim 7, wherein said restoring ring has a metallic memory that said restoring ring returns to an original shape to open up said opening of said capturing pocket.

10. The vessel recanalizer, as recited in claim 4, wherein said restoring ring is made of nitinol.

11. The vessel recanalizer, as recited in claim 7, wherein said restoring ring is made of nitinol.

12. The vessel recanalizer, as recited in claim 9, wherein said restoring ring is made of nitinol.

13. The vessel recanalizer, as recited in claim 1, wherein said capturing pocket further has a plurality of pores for allowing blood passing therethrough to ensure said blood clog to be captured in said capturing pocket when said capturing pocket is slidably pulled backward.

14. The vessel recanalizer, as recited in claim 2, wherein said capturing pocket further has a plurality of pores for allowing blood passing therethrough to ensure said blood clog to be captured in said capturing pocket when said capturing pocket is slidably pulled backward.

15. The vessel recanalizer, as recited in claim [symbol] , wherein said capturing pocket further has a plurality of pores for allowing blood passing therethrough to ensure said blood clog to be captured in said capturing pocket when said capturing pocket is slidably pulled backward.

16. The vessel recanalizer, as recited in claim 12, wherein said capturing pocket further has a plurality of pores for allowing blood passing therethrough to ensure said blood clog to be captured in said capturing pocket when said capturing pocket is slidably pulled backward.

17. The vessel recanalizer, as recited in claim 1, wherein said capturing pocket has a tapered shape reducing a size from said opening for reinforcing said blood clog to be captured in said capturing pocket when said capturing pocket is slidably pulled backward.

18. The vessel recanalizer, as recited in claim 5, wherein said capturing pocket has a tapered shape reducing a size from said opening for reinforcing said blood clog to be captured in said capturing pocket when said capturing pocket is slidably pulled backward.

19. The vessel recanalizer, as recited in claim 12, wherein said capturing pocket has a tapered shape reducing a size from said opening for reinforcing said blood clog to be captured in said capturing pocket when said capturing pocket is slidably pulled backward.

20. The vessel recanalizer, as recited in claim 16, wherein said capturing pocket has a tapered shape reducing a size from said opening for reinforcing said blood clog to be captured in said capturing pocket when said capturing pocket is slidably pulled backward.

21. The vessel recanalizer, as recited in claim 1, wherein said main wire further has a floppy end portion formed at said guiding end towards said capturing pocket for allowing said main wire to extend along said blood vessel until said capturing pocket reaches said blood clog.

22. The vessel recanalizer, as recited in claim 4, wherein said main wire further has a floppy end portion formed at said guiding end towards said capturing pocket for allowing said main wire to extend along said blood vessel until said capturing pocket reaches said blood clog.

23. The vessel recanalizer, as recited in claim 16, wherein said main wire further has a floppy end portion formed at said guiding end towards said capturing pocket for allowing said main wire to extend along said blood vessel until said capturing pocket reaches said blood clog.

24. The vessel recanalizer, as recited in claim 20, wherein said main wire further has a floppy end portion formed at said guiding end towards said capturing pocket for allowing said main wire to extend along said blood vessel until said capturing pocket reaches said blood clog.

25. The vessel recanalizer, as recited in claim 1, wherein said guiding end is covered by platinum.

26. The vessel recanalizer, as recited in claim 12, wherein said guiding end is covered by platinum.

27. The vessel recanalizer, as recited in claim 24, wherein said guiding end is covered by platinum.

28. A method for removing blood clog in blood vessel by a vessel recanalizer which comprises a main wire and a capturing pocket provided thereon, comprising the steps of:
(a) slidably inserting the vessel recanalizer along said blood vessel until said capturing pocket is positioned in vicinity of said blood clog;
(b) opening said capturing pocket and aligning an opening thereof towards said blood clog;
(c) capturing said blood clog in said capturing pocket;
(d) closing said opening of said capturing pocket to trap said piece of blood clog therein; and
(e) slidably ejecting said main wire from said blood vessel to remove said blood clog therein.

29. The method as recited in claim 28, in step (b), further comprising the steps of:
(b.1) slidably pulling said introducer catheter backward against said main wire until said capturing pocket is slid out thereof to open said opening of said capturing pocket, wherein said main wire is slidably received in said introducer catheter to retain said capturing pocket in a closed position while said introducer catheter is slid along said blood vessel to locate said blood clog; and
(b.2) expanding a restoring ring at said opening of said capturing pocket to fully open said opening thereof.

30. The method, as recited in claim 29, in step (d), further comprising the steps of:
(d.1) slidably pulling said main wire along said introducer catheter in a backward direction;
(d.2) contracting said restoring ring against said introducer catheter to enclose said blood clog in said capturing pocket; and
(d.3) slidably pulling said main wire and introducer catheter along said retriever sheath until said capturing pocket is slidably received in said retriever sheath.

31. The method, as recited in claim 30, wherein said restoring ring is made of thermo sensitive material that said restoring ring increases a stiffness thereof at a body temperature.

32. The method, as recited in claim 30, wherein said restoring ring has a metallic memory that said restoring ring returns to an original shape to open up said opening of said capturing pocket.

33. The method, as recited in claim 31, wherein said restoring ring has a metallic memory that said restoring ring returns to an original shape to open up said opening of said capturing pocket.

34. The method, as recited in claim 30, wherein said restoring ring is made of nitinol.

35. The method, as recited in claim 33, wherein said restoring ring is made of nitinol.

36. The method as recited in claim 28, in step (a), wherein said main wire is slid along said blood vessel until said opening of said capturing pocket passes said blood clog.

37. The method as recited in claim 30, in step (a), wherein said main wire is slid along said blood vessel until said opening of said capturing pocket passes said blood clog.

38. The method, as recited in claim 28, in step (c), further comprising the steps of:
(c.1) guiding blood to flow out of said capturing pocket through a plurality of pore provided thereon when said blood clog is captured in said capturing pocket; and
(c.2) ensuring said blood clog to be trapped at a rear portion of said capturing pocket when closing said opening of said capturing pocket.

39. The method as recited in claim 30, in step (c), further comprising the steps of:
(c.1) guiding blood to flow out of said capturing pocket through a plurality of pore provided thereon when said blood clog is captured in said capturing pocket; and
(c.2) ensuring said piece of blood clog to be trapped at a rear portion of said capturing pocket when closing said opening of said capturing pocket.

40. The method as recited in claim 37, in step (c), further comprising the steps of:
(c.1) guiding blood to flow out of said capturing pocket through a plurality of pore provided thereon when said blood clog is captured in said capturing pocket; and
(c.2) ensuring said piece of blood clog to be trapped at a rear portion of said capturing pocket when closing said opening of said capturing pocket.
